(19) United States
(12) Patent Application Publication
(10) Pub. No.: US 2003/0084475 A1
(43) Pub. Date: May 1, 2003

(54) Nucleic acid fragments and proteins affecting storage organelle formation and methods of use

(76) Inventors: Edgar B. Cahoon, (US); Sean J. Coughlan, (US); Timothy George Helentjaris, (US); Rudolf Jung, (US); Chun Ping Li, Johnston, IA (US); Scott E. Nichols, Johnston, IA (US); Kevin G. Ripp, Wilmington, DE (US); Peizhong Zheng, Johnston, IA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/268,441

(22) Filed: Oct. 9, 2002

Related U.S. Application Data
(63) Continuation of application No. 09/672,607, filed on Sep. 28, 2000.
(60) Provisional application No. 60/157,209, filed on Sep. 30, 1999.

(51) Int. Cl. A01H 1/00; C07H 21/04; C12P 21/02; C12N 9/50; C12N 5/04; C12N 5/10

(52) U.S. Cl. 800/278; 435/69.1; 435/219; 435/320.1; 435/419; 536/23.2; 504/116.1; 435/317.1

(57) ABSTRACT

This invention relates to an isolated nucleic acid fragment encoding an SSE1 protein. The invention also relates to the construction of a chimeric gene encoding all or a portion of the SSE1 protein, in sense or antisense orientation, wherein expression of the chimeric gene results in production of altered levels of the SSE1 protein in a transformed host cell. The present invention also relates to methods using the SSE1 protein in modulating formation of storage organelles and storage compounds in seeds, and in discovering compounds with potential herbicidal activity.

This application claims the benefit of U.S. Provisional Application No. 60/157209, filed Sep. 30, 1999.

### FIELD OF THE INVENTION

This invention is in the field of plant molecular biology. More specifically, this invention pertains to nucleic acid fragments encoding SSE1 homologs in plants and seeds.

### BACKGROUND OF THE INVENTION

Seeds of flowering plants contain proteins, starches, and oils in a balance suitable to support seedling growth of the next generation. Users of seeds for food, feed, or industrial purposes often desire modification in quality or quantity of these components.

For example, feed formulations based on crop plants often must be supplemented with specific amino acids to provide animals with essential nutrients which are necessary for their growth. This supplementation is necessary because, in general, crop plants contain low proportions of several amino acids which are essential for, and cannot be synthesized by, monogastric animals. Among the amino acids necessary for animal nutrition, those that are of limited availability in crop plants include methionine, lysine, and threonine.

Attempts to increase the levels of these amino acids by breeding and mutant selection have met with limited success or have been accompanied by a loss in yield. For example, although seeds of corn plants containing a mutant transcription factor (opaque-2) or a mutant α-zein gene (floury-2) exhibit elevated levels of total and bound lysine, there is an altered seed endosperm structure which is more susceptible to insects, pathogens, and mechanical damage. Significant yield losses are also typical. (Glover, D. V., and E. T. Mertz, “Corn” in: Olson and Frey (eds.), Nutritional Quality of Cereal Grains: Genetic and Agronomic Improvement (Madison, Wis., American Society of Agronomy) Agronomy Monograph 28:183-336.)

Grain quality traits are usually quantitatively inherited, and their transfer into commercially acceptable genetic material often requires many generations. Therefore, there is a need for a more direct way of modifying seed quality. As more becomes known about seed storage proteins and the expression of the genes which encode these proteins, and as transformation systems are developed for a greater variety of plants, molecular approaches for improving the nutritional quality of seed proteins can provide alternatives to the more conventional approaches.

For instance, recombinant DNA and gene transfer technologies have been applied to alter enzyme activity at key steps in the amino acid biosynthetic pathway. The introduction into plants of a feedback-regulation-insensitive dihydrodipicolinic acid synthase (“DHDPS”) gene, which encodes an enzyme that catalyzes the first reaction unique to the lysine biosynthetic pathway, has resulted in an increase in the levels of free lysine in the leaves and seeds of those plants (Falco, U.S. Pat. No. 5,773,691; Glassman, U.S. Pat. No. 5,258,300). Also, expression in plants of a bacterial lysC gene with aspartate kinase activity has resulted in an increase in threonine content of the seed (Karchi, et al. The Plant J. 3:721-727 (1993); Galili, et al., European Patent Application No. 0485970). However, expression of the lysC gene results in only a 6-7% increase in the level of total threonine or methionine in the seed; thus, feed containing lysC transgenic seeds still requires amino acid supplementation.

Moreover, modification of the amino acid levels in seeds is not always correlated with changes in the level of proteins that incorporate those amino acids. See Burrow, et al., Molecular & General Genetics Vol. 241; pp. 431-439; (1993). In another study (Falco et al., Biotechnology 13:577-582, 1995), manipulation of bacterial DHDPS and aspartate kinase did result in useful increases in free lysine and total seed lysine. However, abnormal accumulation of lysine catabolites was also observed, suggesting that the free lysine pool was subject to catabolism.

Another alternative method is to express a heterologous protein of favorable amino acid composition at levels sufficient to obviate feed supplementation. As an example, tobacco has been transformed with a chimeric gene containing the bean phaseolin promoter and the cDNA of the sulfur-rich Brazil Nut Protein (“BNP”, 18 mol % methionine and 8 mol % cysteine) into tobacco. Amino acid analysis indicates that the methionine content in the transgenic seeds is enhanced by 30% over that of the untransformed seeds. However, even though BNP increases the amount of total methionine and bound methionine, thereby improving nutritional value, there appears to be a threshold limitation as to the total amount of methionine that is accumulated in the seeds; methionine-rich BNP may be made at the expense of endogenous sulfur-containing compounds. This same chimeric gene has also been transferred into canola, and similar levels of enhancement were achieved. However, an adverse effect is that lysine content decreases. Finally, BNP has been identified as a major food allergen. Thus it is neither practical nor desirable to use BNP to enhance the nutritional value of crop plants (Saalbach, et al., Molecular and General Genetics 242(2):226-236 (1994); Melo-Vania, et al., Food and Agricultural Immunology 6(2):185-195 (1994); Higgins, T. J., et al., WO 99/15004 (1999).

Transformation with a heterologous protein of favorable amino acid composition may also result in pleiotropy. Higgins et al. (WO 99/15004) recently attempted to increase the nutritive value of plant storage organs by transforming legumes with a sunflower seed albumin gene. In addition to the expected effects on sulfur-rich protein content, there were unexpected effects on other quality traits, including total protein content, fiber composition, oil content and composition, starch content, and anti-nutritional factors.

In view of these efforts, it is clear that there continues to be a need for a method to improve nutritional content of seeds by altering protein content.

Starches are polymers of glucose molecules produced and stored only in the chloroplasts and amyloplasts of plants. Most of the starch produced in the world is used as food, but about one-third of the total production is employed for a variety of industrial purposes that take advantage of starch's unique properties. These properties (e.g. viscosity, gelatinization temperature) vary greatly with the plant source and affect the usefulness of the starch for food and nonfood products (Sivak and Preiss, Advances in Food and Nutrition Research, Vol. 41. Academic Press, 1998, p. 163). Starches with desirable functional properties may be currently available only in small quantities, or in plants or plant parts not commonly processed. Therefore, there is a need for a method to alter the amount of starch synthesized and stored in plant seeds.

As to oils, the third major grain quality component, traditional breeding methods have been successful in producing maize populations with extremely high or extremely low levels of oil in the seed (Dudley, J. W., ed. Seventy Generations of Selection for Oil and Protein in Maize. Madison Wis., Crop Science Society of America, 1974). Another method for producing grain with high oil content is provided by Bergquist et al. (U.S. Pat. No. 5,706,603). However, that method is intended for use in maize production fields, giving rise to heterogeneous kernels suitable for feeding or processing; it would not be appropriate for breeders developing inbred lines of maize, nor would it be useful in other species in which cross-pollination is not so easily manipulated. Thus a need persists for a method to alter the amount of oil in plant seeds.

There is a need for methods to improve the nutritional value of plant seeds through genetic modification not accompanied by detrimental side effects such as allergenicity, anti-nutritional quality, or poor yield. A method for modulating the balance of protein, oil, and starch components in the seed by altering the relative partitioning of photosynthate to each seed component would therefore be of interest.

An interesting possibility lies in the use of the SSE1 gene which encodes a protein that shares homology with the peripheral peroxisomal membrane protein PEX16 in Yarrowia lipolytica which has been shown to be important for peroxisome assembly (Eitzen et al. (1997) J Cell Biol 137:1265-1278; Lin et al. (1999) Science 284:328-330). SSE1 complements a pex16 mutation as indicated by growth of the transformed mutant cell on oleic acid as sole carbon source; pex16 mutants are unable to grow on oleic acid since they are defective in assembling peroxisomes, which are solely able to metabolize oleic acid.

The SSE1 gene has only been cloned from Arabidopsis (Lin et al. (1999) Science 284:328-330). Homozygous sse1 (shrunken seed 1) mutant Arabidopsis seeds were not viable, and accumulated starch instead of mainly proteins and lipids which are the major storage compounds in mature Arabidopsis seeds. This suggests that SSE1 is needed for the pathway leading to seed storage protein and lipid deposition, and if said pathway is inactivated, seed starch formation proceeds by default. Accordingly, profile of seed storage compounds may be altered by modulating SSE1 expression. For example, protein content of cereal grains which predominantly accumulate starch may be enhanced by overexpressing SSE1.

### SUMMARY OF THE INVENTION

The instant invention relates to isolated nucleic acid fragments encoding SSE1 homologs. Specifically, this invention concerns an isolated nucleic acid fragment encoding an SSE1 protein and an isolated nucleic acid fragment that is substantially similar to an isolated nucleic acid fragment encoding an SSE1 protein. In addition, this invention relates to a nucleic acid fragment that is complementary to the nucleic acid fragment encoding an SSE1 protein.

An additional embodiment of the instant invention pertains to a polypeptide encoding all or a substantial portion of an SSE1 protein.

In another embodiment, the instant invention relates to a chimeric gene encoding an SSE1 protein, or to a chimeric gene that comprises a nucleic acid fragment that is complementary to a nucleic acid fragment encoding an SSE1 protein, operably linked to suitable regulatory sequences, wherein expression of the chimeric gene results in production of levels of the encoded protein in a transformed host cell that is altered (i.e., increased or decreased) from the level produced in an untransformed host cell.

In a further embodiment, the instant invention concerns a transformed host cell comprising in its genome a chimeric gene encoding an SSE1 protein, operably linked to suitable regulatory sequences. Expression of the chimeric gene results in production of altered levels of the encoded protein in the transformed host cell. The transformed host cell can be of eukaryotic or prokaryotic origin, and include cells derived from higher plants and microorganisms. The invention also includes transformed plants that arise from transformed host cells of higher plants, and seeds derived from such transformed plants.

An additional embodiment of the instant invention concerns a method of altering the level of expression of an SSE1protein in a transformed host cell comprising: (a) transforming a host cell with a chimeric gene comprising a nucleic acid fragment encoding an SSE1 protein; and (b) growing the transformed host cell under conditions that are suitable for expression of the chimeric gene wherein expression of the chimeric gene results in production of altered levels of SSE1 protein in the transformed host cell.

An addition embodiment of the instant invention concerns a method for obtaining a nucleic acid fragment encoding all or a substantial portion of an amino acid sequence encoding an SSE1 protein.

In a further embodiment, the instant invention concerns a method of modulating expression of SSE1 and/or homologs thereof in a plant, comprising the steps of: (a) transforming a plant cell with a nucleic acid fragment comprising the SSE1 protein operably linked to a promoter in sense or antisense orientation; and (b) growing the plant cell under plant growing conditions to produce a regenerated plant capable of expressing the nucleic acid for a time sufficient to modulate expression of the nucleic acid fragment in the plant compared to a corresponding non-transformed plant, thereby resulting in at least one of the following: modulating the relative amounts of oil, protein and/or starch in the seed of a plant, modulating storage organ formation in the seed of a plant, improving the food, feed, and/or industrial processing value of grain, and partitioning photosynthate to produce seed with improved functional properties for use in specific food and non-food industrial applications.

The present invention relates to isolated polynucleotides comprising a nucleotide sequence encoding a first polypeptide of at least 50 amino acids that has at least 80% identity based on the Clustal method of alignment when compared to a polypeptide selected from the group consisting of a corn SSE1 polypeptide of SEQ ID NO: 2, a rice SSE1 polypeptide of SEQ ID NO: 4, a rice SSE1 polypeptide of SEQ ID NO: 6, a soybean SSE1 polypeptide of SEQ ID NO: 8, a soybean SSE1 polypeptide of SEQ ID NO: 10, a wheat SSE1 polypeptide of SEQ ID NO: 12, and a wheat SSE1 polypeptide of SEQ ID NO: 14. The present invention also relates to an isolated polynucleotide comprising the complement of the nucleotide sequences described above.

It is preferred that the isolated polynucleotides of the claimed invention comprises a nucleic acid sequence selected from the group consisting of SEQ ID NOs: 1, 3, 5, 7, 9, 11, and 13 that codes for the polypeptide selected from the group consisting of SEQ ID NOs: 2, 4, 6, 8, 10, 12, and 14. The present invention also relates to an isolated polynucleotide comprising a nucleotide sequences of at least one of 40 (preferably at least one of 30, most preferred at least one of 15) contiguous nucleotides derived from a nucleotide sequence selected from the group consisting of SEQ ID NOs: 1, 3, 5, 7, 9, 11, and 13 and the complement of such nucleotide sequences.

The present invention relates to a chimeric gene comprising an isolated polynucleotide of the present invention operably linked to at least one suitable regulatory sequence.

The present invention relates to an isolated host cell comprising a chimeric gene of the present invention or an isolated polynucleotide of the present invention. The host cell may be eucaryotic, such as a yeast or a plant cell, or procaryotic, such as a bacterial cell. The present invention also relates to a host cell such as a virus, preferably a baculovirus, comprising an isolated polynucleotide of the present invention or a chimeric gene of the present invention.

The present invention relates to a process for producing an isolated host cell comprising a chimeric gene of the present invention or an isolated polynucleotide of the present invention, the process comprising either transforming or transfecting an isolated compatible host cell with a chimeric gene or isolated polynucleotide of the present invention.

The present invention relates to a polypeptide of at least 50 amino acids comprising at least 80% homology based on the Clustal method of alignment compared to an SSE1 polypeptide selected from the group consisting of SEQ ID NOs: 2, 4, 6, 8, 10, 12, and 14.

The present invention relates to a method of selecting an isolated polynucleotide that affects the level of expression of an SSE1 polypeptide or enzyme activity in a host cell, preferably a plant cell, the method comprising the steps of: (a) constructing an isolated polynucleotide of the present invention or an isolated chimeric gene of the present invention; (b) introducing the isolated polynucleotide or the isolated chimeric gene into a host cell; (c) measuring the level of the polypeptide or enzyme activity in the host cell containing the isolated polynucleotide; and (d) comparing the level of the SSE1 polypeptide or enzyme activity in the host cell containing the isolated polynucleotide with the level of the SSE1 polypeptide or enzyme activity in the host cell that does not contain the isolated polynucleotide.

The present invention relates to a method of obtaining a nucleic acid fragment encoding a substantial portion of an SSE1 polypeptide, preferably a plant SSE1 polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs: 1, 3, 5, 7, 9, 11, and 13 and the complement of such nucleotide sequences; and amplifying a nucleic acid fragment (preferably a cDNA inserted in a cloning vector) using the oligonucleotide primer. The amplified nucleic acid fragment preferably will encode a portion of an SSE1 amino acid sequence.

The present invention also relates to a method of obtaining a nucleic acid fragment encoding all or a substantial portion of the amino acid sequence encoding an SSE1 polypeptide comprising the steps of: probing a cDNA or genomic library with an isolated polynucleotide of the present invention; identifying a DNA clone that hybridizes with an isolated polynucleotide of the present invention; isolating the identified DNA clone; and sequencing the cDNA or genomic fragment that comprises the isolated DNA clone.

This invention also concerns a composition, such as a hybridization mixture, comprising an isolated polynucleotide of the present invention.

The present invention also relates to a method for positive selection of a transformed cell comprising: (a) transforming a host cell with the chimeric gene of the present invention or an expression cassette of the present invention; and (b) growing the transformed host cell, preferably a plant cell, such as a monocot or a dicot, under conditions which allow expression of the SSE1 polynucleotide in an amount sufficient to complement a null mutant to provide a positive selection means.

This invention also concerns a method of altering the level of expression of SSE1 protein in a host cell comprising: (a) transforming a host cell with a chimeric gene of the present invention; and (b) growing the transformed host cell under conditions that are suitable for expression of the chimeric gene wherein expression of the chimeric gene results in production of altered levels of the SSE1 protein in the transformed host cell.

### BRIEF DESCRIPTION OF THE DRAWING AND SEQUENCE DESCRIPTIONS

The invention can be more fully understood from the following detailed description and the accompanying drawing and Sequence Listing which forms a part of this application.

FIG. 1 depicts the amino acid sequence alignment between the SSE1 protein encoded by the nucleotide sequences derived from maize clone p0002.cgevh96r (SEQ ID NO: 2), rice clone rl0n.pk0031.h7 (SEQ ID NO: 6), contig assembled from soybean clones sdp3c.pk008.c9 and pP54/pP55 (SEQ ID NO: 10), wheat clone wdk5c.pk0002.b10 (SEQ ID NO: 14), and the SSE1 gene from Arabidopsis thaliana (NCBI GenBank Identifier (GI) No. 4837733, SEQ ID NO: 15). Amino acids which are conserved among all and at least two sequences with an amino acid at that position are indicated with an asterisk (*). Dashes are used by the program to maximize alignment of the sequences.

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. Table 1 also identifies the cDNA clones as individual ESTs (“EST”), sequences of the entire cDNA inserts comprising the indicated cDNA clones (“FIS”), sequences of contigs assembled from two or more ESTs (“Contig”), sequences of contigs assembled from an FIS and one or more ESTs or PCR fragment sequence (“Contig*”), or sequences encoding the entire protein derived from an FIS, a contig, or an FIS and PCR fragment sequence (“CGS”). Nucleotide SEQ ID NOs: 1, 3, 7, and 11 correspond to nucleotide SEQ ID NOs: 1, 3, 5, and 7, respectively, presented in U.S. Provisional Application No. 60/157209, filed Sep. 30, 1999. Amino acid SEQ ID NOs: 2, 4, 8, and 12 correspond to amino acid SEQ ID NOs: 2, 4, 6, and 8, respectively, presented in U.S. Provisional Application No. 60/157209, filed Sep. 30, 1999. The sequence descriptions and Sequence Listing attached hereto comply with the rules governing nucleotide and/or amino acid sequence disclosures in patent applications as set forth in 37 C.F.R. §1.821-1.825.

TABLE 1
SSE1 Homologs

| | | | SEQ ID NO: | |
|---|---|---|---|---|
| Protein | | | (Nucleo- | (Amino |
| (Plant Source) | Clone Designation | Status | tide) | Acid) |
| SSE1 (Maize) | p0002.cgevh96r (FIS) | CGS | 1 | 2 |
| SSE1 (Rice) | r10n.pk0031.h7 | EST | 3 | 4 |
| SSE1 (Rice) | r10n.pk0031.h7 (FIS) | CGS | 5 | 6 |
| SSE1 | Contig of | CGS | 7 | 8 |
| (Soybean) | sdp3c.pk003.e23 | | | |
| | sdp3c.pk008.c9 | | | |
| | pP54/pP55 | | | |
| SSE1 | Contig of | CGS | 9 | 10 |
| (Soybean) | sdp3c.pk008.c9 (FIS) | | | |
| | pP54/pP55 | | | |
| SSE1 (Wheat) | wdk5c.pk0002.b10 | EST | 11 | 12 |
| SSE1 (Wheat) | wdk5c.pk0002.b10 (FIS) | CGS | 13 | 14 |

SEQ ID NO: 15 is the amino acid sequence of the polypeptide encoded by the SSE1 gene from Arabidopsis thaliana (NCBI GenBank Identifier (GI) No. 4837733).

SEQ ID NO: 16 sets forth the sequence of an oligonucleotide that may be used as probe in library subtraction.

SEQ ID NOS: 17 and 18 set forth the sequence of PCR primers used in amplifying a portion of the soybean SSE1 nucleic acid fragment.

The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

In the context of this disclosure, a number of terms shall be utilized. The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. An isolated polynucleotide of the present invention may include at least one of 60 contiguous nucleotides, preferably at least one of 40 contiguous nucleotides, most preferably one of at least 30 contiguous nucleotides derived from SEQ ID NOs: 1, 3, 5, 7, 9, 11, or 13, or the complement of such sequences.

The term “isolated” polynucleotide refers to a polynucleotide that is substantially free from other nucleic acid sequences, such as other chromosomal and extrachromosomal DNA and RNA, that normally accompany or interact with it as found in its naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

Substantially similar nucleic acid fragments may be selected by screening nucleic acid fragments representing subfragments or modifications of the nucleic acid fragments of the instant invention, wherein one or more nucleotides are substituted, deleted and/or inserted, for their ability to affect the level of the polypeptide encoded by the unmodified nucleic acid fragment in a plant or plant cell. For example, a substantially similar nucleic acid fragment representing at least one of 30 contiguous nucleotides derived from the instant nucleic acid fragment can be constructed and introduced into a plant or plant cell. The level of the polypeptide encoded by the unmodified nucleic acid fragment present in a plant or plant cell exposed to the substantially similar nucleic fragment can then be compared to the level of the polypeptide in a plant or plant cell that is not exposed to the substantially similar nucleic acid fragment.

For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Consequently, a polynucleotide comprising a nucleotide sequence of at least one of 60 (preferably at least one of 40, most preferred at least one of 30) contiguous nucleotides derived from a nucleotide sequence selected from the group consisting of SEQ ID NOs: 1, 3, 5, 7, 9, 11, and 13 and the complement of such nucleotide sequences may be used in methods of selecting an isolated polynucleotide that affects the expression of a polypeptide (such as SSE1) in a plant cell. A method of selecting an isolated polynucleotide that affects the level of expression of a polypeptide in a virus or in a host cell (eukaryotic, such as plant or yeast, prokarotic such as bacterial) may comprise the steps of: constructing an isolated polynucleotide of the present invention or an isolated chimeric gene of the present invention; introducing the isolated polynucleotide or the isolated chimeric gene into a host cell; measuring the level a polypeptide or enzyme activity in the host cell containing the isolated polynucleotide; and comparing the level of a polypeptide or enzyme activity in the host cell containing the isolated polynucleotide with the level of a polypeptide or enzyme activity in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6× SSC, 0.5% SDS at room temperature for 15 min, then repeated with 2× SSC, 0.5% SDS at 45° C. for 30 min, and then repeated twice with 0.2× SSC, 0.5% SDS at 50° C. for 30 min. A more preferred set of stringent conditions uses higher temperatures in which the washes are identical to those above except for the temperature of the final two 30 min washes in 0.2× SSC, 0.5% SDS was increased to 60° C. Another preferred set of highly stringent conditions uses two final washes in 0.1× SSC, 0.1% SDS at 65° C.

Substantially similar nucleic acid fragments of the instant invention may also be characterized by the percent identity of the amino acid sequences that they encode to the amino acid sequences disclosed herein, as determined by algorithms commonly employed by those skilled in this art. Generally, substantially similar nucleic acid fragments encode amino acid sequences that are at least 50% identical to the amino acid sequences reported herein. Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids. Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

As used herein, “SSE1 homolog” refers to either polypeptide or nucleic acid fragment, or both, that is substantially similar to the SSE1 gene or gene product of Arabidopsis thaliana.

As used herein, “SSE1protein” refers to either the polypeptide encoded by the SSE1 gene of Arabidopsis thaliana or a polypeptide encoded by another SSE1 homolog, or both. “SSE1 protein” and “SSE1 polypeptide” are used interchangeably herein.

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST (Basic Local Alignment Search Tool; Altschul et al. (1993) J. Mol. Biol. 215:403-410; see also www.ncbi.nlm.nih.gov/BLAST/). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available. “Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

The “translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Molecular Biotechnology 3:225).

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into polypeptide by the cell. “cDNA” refers to DNA that is complementary to and derived from mRNA. The cDNA can be single-stranded or converted to double stranded form using, for example, the Klenow fragment of DNA polymerase I. “Sense” RNA refers to an RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

A “protein” or “polypeptide” is a chain of amino acids arranged in a specific order determined by the coding sequence in a polynucleotide encoding the polypeptide.

“Altered levels” refers to the production of gene product(s) in transgenic organisms in amounts or proportions that differ from that of normal or non-transformed organisms.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Transformed plant cells which are derived by any of the above transformation techniques can be cultured to regenerate a whole plant which possesses the transformed genotype. Such regeneration techniques often rely on manipulation of certain phytohormones in a tissue culture growth medium, typically relying on a biocide and/or herbicide marker which has been introduced together with a nucleic acid fragment of the present invention. For transformation and regeneration of maize see, Gordon-Kamm et al., The Plant Cell, 2:603-618 (1990).

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch et al., Science, 227:1229-1231 (1985) and Fraley et al., Proc. Natl. Acad. Sci. USA 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee et al., Ann. Rev. of Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see generally, The Maize Handbook, Freeling and Walbot, Eds., Springer, N.Y. (1994); Corn and Corn Improvement, 3^{rd} edition, Sprague and Dudley Eds., American Society of Agronomy, Madison, Wis. (1988).

One of skill will recognize that after the transgene/s is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed-propagated crops, mature transgenic plants can be self crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a selectable marker can be screened for transmission of the nucleic acid of the present invention by, for example, standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific nucleic acid fragment probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a nucleic acid fragment of the present invention relative to a control plant (i.e., native, non-transgenic). Back-crossing to a parental plant and out-crossing with a non-transgenic plant are also contemplated.

The present invention provides a method of genotyping a plant comprising a nucleic acid fragment of the present invention. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in Plants (ed. Andrew H. Paterson) by Academic Press/R. G. Landis Company, Austin, Tex. pp. 7-21.

The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide sequence variability. Thus, the present invention further provides a means to follow segregation of a gene or nucleic acid of the present invention as well as chromosomal sequences genetically linked to these genes or nucleic acids using such techniques as RFLP analysis.

Plants that can be transformed in the method of the invention include monocotyledonous and dicotyledonous plants. Preferred plants include maize, soybean, alfalfa, sunflower, canola, cotton, palm, flax, sorghum, wheat, barley, millet, and rice.

Seeds derived from plants regenerated from transformed plant cells, plant parts or plant tissues, or progeny derived from the regenerated transformed plants, may be used directly as feed or food, or further processing may occur.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Maniatis”).

Nucleic acid fragments encoding at least a portion of several SSE1 homologs have been isolated and identified by comparison of random plant cDNA sequences to public databases containing nucleotide and protein sequences using the BLAST algorithms well known to those skilled in the art. The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other SSE1 homologs, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, the entire sequences can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction (PCR) protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al. (1988) Proc. Natl. Acad. Sci. USA 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al. (1989) Proc. Natl. Acad. Sci. USA 86:5673; Loh et al. (1989) Science 243:217). Products generated by the 3′ and 5′ RACE procedures can be combined to generate full-length cDNAs (Frohman and Martin (1989) Techniques 1:165). Consequently, a polynucleotide comprising a nucleotide sequence of at least one of 60 (preferably one of at least 40, most preferably one of at least 30) contiguous nucleotides derived from a nucleotide sequence selected from the group consisting of SEQ ID NOs: 1, 3, 5, 7, 9, 11, and 13 and the complement of such nucleotide sequences may be used in such methods to obtain a nucleic acid fragment encoding a substantial portion of an amino acid sequence of a polypeptide (such as SSE1).

The present invention relates to a method of obtaining a nucleic acid fragment encoding a substantial portion of an SSE1 polypeptide, preferably a substantial portion of a plant SSE1 polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs: 1, 3, 5, 7, 9, 11, and 13 and the complement of such nucleotide sequences; and amplifying a nucleic acid fragment (preferably a cDNA inserted in a cloning vector) using the oligonucleotide primer. The amplified nucleic acid fragment preferably will encode a portion of an SSE1 polypeptide.

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner (1984) Adv. Immunol. 36: 1; Maniatis).

In another embodiment, this invention concerns viruses and host cells comprising either the chimeric genes of the invention as described herein or an isolated polynucleotide of the invention as described herein. Examples of host cells which can be used to practice the invention include, but are not limited to, yeast, bacteria, and plants.

As was noted above, the nucleic acid fragments of the instant invention may be used to create transgenic plants in which the disclosed polypeptides are present at higher or lower levels than normal or in cell types or developmental stages in which they are not normally found. This would have the effect of altering the level of particular seed storage compounds in those cells.

Overexpression of the proteins of the instant invention may be accomplished by first constructing a chimeric gene in which the coding region is operably linked to a promoter capable of directing expression of a gene in the desired tissues at the desired stage of development. The chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant isolated polynucleotide (or chimeric gene) may be constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant polypeptides to different cellular compartments, or to facilitate its secretion from the cell. It is thus envisioned that the chimeric gene described above may be further supplemented by directing the coding sequence to encode the instant polypeptides with appropriate intracellular targeting sequences such as transit sequences (Keegstra (1989) Cell 56:247-253), signal sequences or sequences encoding endoplasmic reticulum localization (Chrispeels (1991) Ann. Rev. Plant Phys. Plant Mol. Biol. 42:21-53), or nuclear localization signals (Raikhel (1992) Plant Phys. 100: 1627-1632) with or without removing targeting sequences that are already present. While the references cited give examples of each of these, the list is not exhaustive and more targeting signals of use may be discovered in the future.

It may also be desirable to reduce or eliminate expression of genes encoding the instant polypeptides in plants for some applications. In order to accomplish this, a chimeric gene designed for co-suppression of the instant polypeptide can be constructed by linking a gene or gene fragment encoding that polypeptide to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression (U.S. Pat. Nos. 5,190,931, 5,107,065 and 5,283,323). An antisense or cosuppression construct would act as a dominant negative regulator of gene activity. While conventional mutations can yield negative regulation of gene activity these effects are most likely recessive. The dominant negative regulation available with a transgenic approach may be advantageous from a breeding perspective. In addition, the ability to restrict the expression of a specific phenotype to the reproductive tissues of the plant by the use of tissue specific promoters may confer agronomic advantages relative to conventional mutations which may have an effect in all tissues in which a mutant gene is ordinarily expressed.

The person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds. For example, one can screen by looking for changes in gene expression by using antibodies specific for the protein encoded by the gene being suppressed, or one could establish assays that specifically measure enzyme activity. A preferred method will be one which allows large numbers of samples to be processed rapidly, since it will be expected that a large number of transformants will be negative for the desired phenotype.

The instant polypeptides (or portions thereof) may be produced in heterologous host cells, particularly in the cells of microbial hosts, and can be used to prepare antibodies to the these proteins by methods well known to those skilled in the art. The antibodies are useful for detecting the polypeptides of the instant invention in situ in cells or in vitro in cell extracts. Preferred heterologous host cells for production of the instant polypeptides are microbial hosts. Microbial expression systems and expression vectors containing regulatory sequences that direct high level expression of foreign proteins are well known to those skilled in the art. Any of these could be used to construct a chimeric gene for production of the instant polypeptides. This chimeric gene could then be introduced into appropriate microorganisms via transformation to provide high level expression of the encoded SSE1 homologs. An example of a vector for high level expression of the instant polypeptides in a bacterial host is provided (Example 6).

The present invention further provides a method for modulating (i.e., increasing or decreasing) the concentration or composition of the polypeptides of the present invention in a plant or part thereof. Modulation of the polypeptides can be effected by increasing or decreasing the concentration and/or the composition of the polypeptides in a plant. The method comprises transforming a plant cell with an expression cassette comprising a nucleic acid fragment of the present invention to obtain a transformed plant cell, growing the transformed plant cell under plant forming conditions, and expressing the nucleic acid fragment in the plant for a time sufficient to modulate concentration and/or composition of the polypeptides in the plant or plant part.

In some embodiments, the content and/or composition of polypeptides of the present invention in a plant may be modulated by altering, in vivo or in vitro, the promoter of a non-isolated gene of the present invention to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling et al., PCT/US93/03868.

In some embodiments, an isolated nucleic acid fragment (e.g., a vector) comprising a promoter sequence is transfected into a plant cell. Subsequently, a plant cell comprising the isolated nucleic acid is selected for by means known to those of skill in the art such as, but not limited to, Southern blot, DNA sequencing, or PCR analysis using primers specific to the promoter and to the nucleic acid and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art.

In general, concentration of the polypeptides is increased or decreased by at least 5%, 10%, 20%, 30%, 40%, 50%, 60%, 70%, 80%, or 90% relative to a native control plant, plant part, or cell lacking the aforementioned transgene. Modulation in the present invention may occur during and/or subsequent to growth of the plant to the desired stage of development.

Modulating nucleic acid expression temporally and/or in particular tissues can be controlled by employing the appropriate promoter operably linked to a nucleic acid fragment of the present invention in, for example, sense or antisense orientation as discussed in greater detail above. Induction of expression of a nucleic acid fragment of the present invention can also be controlled by exogenous administration of an effective amount of inducing compound. Inducible promoters and inducing compounds that activate expression from these promoters are well known in the art.

Examples of inducible promoters are the Adh1 promoter which is inducible by hypoxia or cold stress, the Hsp70 promoter which is inducible by heat stress, and the PPDK promoter which is inducible by light. Also useful are promoters which are chemically inducible.

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter (Boronat et al. (1986) Plant Sci. 47:95-102; Reina et al. (1990) Nucleic Acids Res. 18(21):6426; Kloesgen et al. (1986) Mol. Gen. Genet. 203:237-244). Promoters that are expressed in the embryo, pericarp, and endosperm are disclosed in U.S. application Ser. Nos. 60/097,233 filed Aug. 20, 1998 and 60/098,230 filed Aug. 28, 1998. The disclosures of each of these are incorporated herein by reference in their entirety.

Either heterologous or non-heterologous (i.e., endogenous) promoters can be employed to direct expression of the nucleic acids of the present invention. These promoters can also be used, for example, in chimeric genes to drive expression of antisense nucleic acids to reduce, increase, or alter concentration and/or composition of the proteins of the present invention in a desired tissue.

All or a substantial portion of the nucleic acid fragments of the instant invention may also be used as probes for genetically and physically mapping the genes that they are a part of, and as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al. (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask (1991) Trends Genet. 7:149-154). Although current methods of FISH mapping favor use of large clones (several to several hundred KB; see Laan et al. (1995) Genome Research 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian (1989) J. Lab. Clin. Med. 114(2):95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nature Genetics 7:22-28) and Happy Mapping (Dear and Cook (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer (1989) Proc. Natl. Acad. Sci USA 86:9402-9406; Koes et al. (1995) Proc. Natl. Acad. Sci USA 92:8149-8153; Bensen et al. (1995) Plant Cell 7:75-84). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the instant polypeptide. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding the instant polypeptide can be identified and obtained. This mutant plant can then be used to determine or confirm the natural function of the instant polypeptides disclosed herein.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Composition of cDNA Libraries: Isolation and Sequencing of cDNA Clones

cDNA libraries representing mRNAs from various maize, rice, soybean and wheat tissues were prepared. The characteristics of the libraries are described below.

TABLE 2
cDNA Libraries from Maize, Rice, Soybean and Wheat

| Library | Tissue | Clone |
|---|---|---|
| p0002 | Maize Tassel: Premeiotic Cells to Early | p0002.cgevh96r |
| | Uninucleate Stage | |
| r10n | Rice 15 Day Old Leaf* | r10n.pk0031.h7 |
| sdp3c | Soybean Developing Pod (8-9 mm) | sdp3c.pk003.e23 |
| | | sdp3c.pk008.c9 |
| se3 | Soybean Embryo, 17 Days After | pP54/pP55** |
| | Flowering | |
| wdk5c | Wheat Developing Kernel, 30 Days After | wdk5c.pk0002.b10 |
| | Anthesis | |

*This library was normalized essentially as described in U.S. Pat. No. 5,482,845, incorporated herein by reference.
**pP54 and pP55 were the resulting plasmids after cloning the PCR products obtained from 5′ RACE PCR using se3 library DNA as template, into pGEM-T Easy (Promega).

Total RNA isolation, Poly(A)+ RNA isolation, and cDNA library construction may be accomplished by any of the many methods available. For example, total RNA may be isolated from plant tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N. Anal. Biochem. 162, 156 (1987)). In brief, plant tissue samples are pulverized in liquid nitrogen before the addition of the TRIzol Reagent, and then are further homogenized with a mortar and pestle. Addition of chloroform followed by centrifugation is conducted for separation of an aqueous phase and an organic phase. The total RNA is recovered by precipitation with isopropyl alcohol from the aqueous phase.

The selection of poly(A)+ RNA from total RNA may be performed using PolyATact system (Promega Corporation. Madison, Wis.). In brief, biotinylated oligo(dT) primers are used to hybridize to the 3′ poly(A) tails on mRNA. The hybrids are captured using streptavidin coupled to paramagnetic particles and a magnetic separation stand. The mRNA is washed at high stringency conditions and eluted by RNase-free deionized water.

cDNA synthesis may be performed and unidirectional cDNA libraries constructed using the SuperScript Plasmid System (Life Technology Inc. Gaithersburg, Md.). The first strand of cDNA is synthesized by priming an oligo(dT) primer containing a Not I site. The reaction is catalyzed by SuperScript Reverse Transcriptase II at 45° C. The second strand of cDNA is labeled with alpha-³²P-dCTP and a portion of the reaction is analyzed by agarose gel electrophoresis to determine cDNA sizes. cDNA molecules smaller than 500 base pairs and unligated adapters are removed by Sephacryl-S400 chromatography. The selected cDNA molecules are ligated into pSPORT1 vector (Life Technology Inc. Gaithersburg, Md.) in between of Not I and Sal I sites.

Alternatively, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products).

Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

cDNA libraries may also be subjected to the subtraction procedure. The libraries are plated out on 22×22 cm2 agar plate at density of about 3,000 colonies per plate. The plates are incubated in a 37° C. incubator for 12-24 hours. Colonies are picked into 384-well plates by a robot colony picker, Q-bot (GENETIX Limited). These plates are incubated overnight at 37° C.

Once sufficient colonies are picked, they are pinned onto 22×22 cm2 nylon membranes using Q-bot. Each membrane contains 9,216 colonies or 36,864 colonies. These membranes are placed onto agar plate with appropriate antibiotic. The plates are incubated at 37° C. for overnight.

After colonies are recovered on the second day, these filters are placed on filter paper prewetted with denaturing solution for four minutes, then are incubated on top of a boiling water bath for additional four minutes. The filters are then placed on filter paper prewetted with neutralizing solution for four minutes. After excess solution is removed by placing the filters on dry filter papers for one minute, the colony side of the filters are placed into Proteinase K solution, incubated at 37° C. for 40-50 minutes. The filters are placed on dry filter papers to dry overnight. DNA is then cross-linked to nylon membrane by UV light treatment.

Colony hybridization is conducted as described by Sambrook, J., Fritsch, E. F. and Maniatis, T., (in Molecular Cloning: A laboratory Manual, 2nd Edition). The following probes are used in colony hybridization:

1. First strand cDNA from the same tissue as the library was made from to remove the most redundant clones.

2. 48-192 most redundant cDNA clones from the same library based on previous sequencing data.

3. 192 most redundant cDNA clones in the entire sequence database.

4. A Sal-A20 oligo nucleotide: 5′-TCG ACC CAC GCG TCC GAA AAA AAA AAA AAA AAA AAA-3′ (SEQ ID NO: 16), which removes clones containing a poly A tail but no cDNA.

5. cDNA clones derived from rRNA.

The image of the autoradiography is scanned into computer and the signal intensity and cold colony addresses of each colony is analyzed. Re-arraying of cold-colonies from 384 well plates to 96 well plates is conducted using Q-bot.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred/Phrap (P. Green, University of Washington, Seattle). Phrep/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (D. Gordon, University of Washington, Seattle).

### Example 2

### Identification of cDNA Clones

cDNA clones encoding SSE1 homologs were identified by conducting BLAST (Basic Local Alignment Search Tool; Altschul et al. (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nature Genetics 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the genbank database as described above. ESTs that contain sequences more 5- or 3-prime can be found by using the BLASTn algorithm (Altschul et al (1997) Nucleic Acids Res. 25:3389-3402.) against the Du Pont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described in Example 1. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 3

### Characterization of cDNA Clones Encoding SSE1 Homologs

The BLASTX search using the EST sequences from clones listed in Table 3 revealed similarity of the polypeptides encoded by the cDNAs to SSE1 protein from Arabidopsis thaliana (NCBI GenBank Identifier (GI) No. 4837733; SEQ ID NO: 15). Shown in Table 3 are the BLAST results for individual ESTs (“EST”), sequences of the entire cDNA inserts comprising the indicated cDNA clones (“FIS”), sequences of contigs assembled from two or more ESTs (“Contig”), sequences of contigs assembled from an FIS and one or more ESTs (“Contig*”), or sequences encoding the entire protein derived from an FIS, a contig, or an FIS and PCR fragment sequence (“CGS”):

TABLE 3
BLAST Results for Sequences Encoding
Polypeptides Homologous to SSE1

| | | BLAST pLog Score |
|---|---|---|
| Clone | Status | 4837733 |
| p0002.cgevh96r (FIS) | CGS | 88.00 |
| r10n.pk0031.h7 | EST | 15.70 |
| r10n.pk0031.h7 (FIS) | CGS | 95.52 |
| Contig of | CGS | 111.00 |
| sdp3c.pk003.e23 | | |
| sdp3c.pk008.c9 | | |
| pP54/pP55 | | |
| Contig of | CGS | 112.00 |
| sdp3c.pk008.c9 (FIS) | | |
| pP54/pP55 | | |
| wdk5c.pk0002.b10 | EST | 10.70 |
| wdk5c.pk0002.b10 (FIS) | CGS | 95.40 |

To generate the full-length coding sequence encoding the soybean SSE1 protein, 5′ RACE PCR was performed. The following oligonucleotides were used as primers:

5′-GCAGACAGATGAAACATTCG-3′ SEQ ID NO: 17

5′-CTCTAGAACTAGTGGATCCC-3′ SEQ ID NO: 18

SEQ ID NO: 17 was based on soybean SSE1 nucleotide sequence obtained from a contig assembled from nucleotide sequences derived from soybean clones sdp3c.pk003.e23 and sdp3c.pk008.c9. SEQ ID NO: 18 was based on sequence of the vector used for library construction. The PCR reaction (100 μl in total volume) consisted of 10 pmoles of the above oligonucleotides, 1 μg se3 library DNA, 200 μM dNTPs, 20 mM Tris-HCl (pH 8.4), 50 mM KCL, 10 mM MgCl₂, 1 unit recombinant Taq DNA polymerase (BRL). PCR was carried out as follows: 4 min at 95° C.; then 30 cycles of: 45 sec at 95° C., 45 sec at 60° C., and 1 min at 72° C.; and finally, 7 min at 72° C. A 1-kb PCR product was purified using Qiagen QIAquick PCR cleanup kit (Qiagen) according to the manufacturer's instructions, and then subcloned into pre-cut pGEM-T Easy (Promega), giving rise to pP54 and pP55. pP54 and pP55 gave slightly different insert sequences, probably in part due to artifacts generated by Taq DNA polymerase. The sequence generated from the PCR product (nucleotides 18 to 1024 in SEQ ID NO: 7) was combined with the nucleotide sequence obtained from a contig assembled from nucleotide sequences derived from soybean clones sdp3c.pk003.e23 and sdp3c.pk008.c9, yielding the full-length coding sequence encoding a soybean SSE1 protein, set forth in SEQ ID NO: 7. Combining the sequence generated from the PCR product (nucleotides 18 to 1024 in SEQ ID NO: 7) with the nucleotide sequence of the entire insert in soybean clone sdp3c.pk008.c9 yields the full-length coding sequence encoding a soybean SSE1 protein with a fewer number of unsure nucleotides than SEQ ID NO: 7, and is set forth in SEQ ID NO: 9.

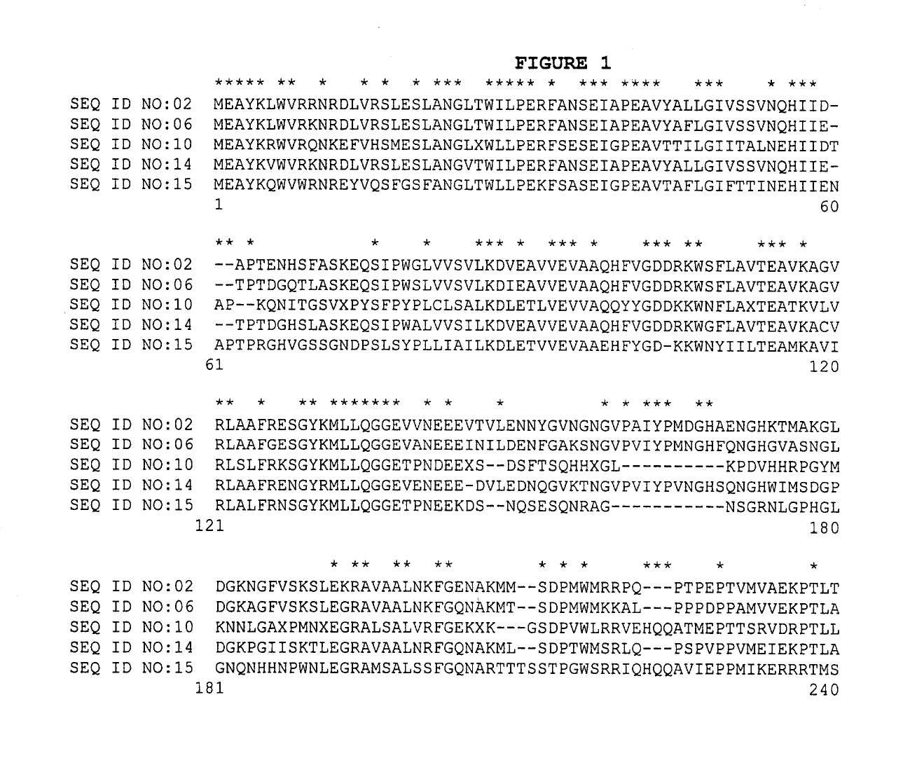

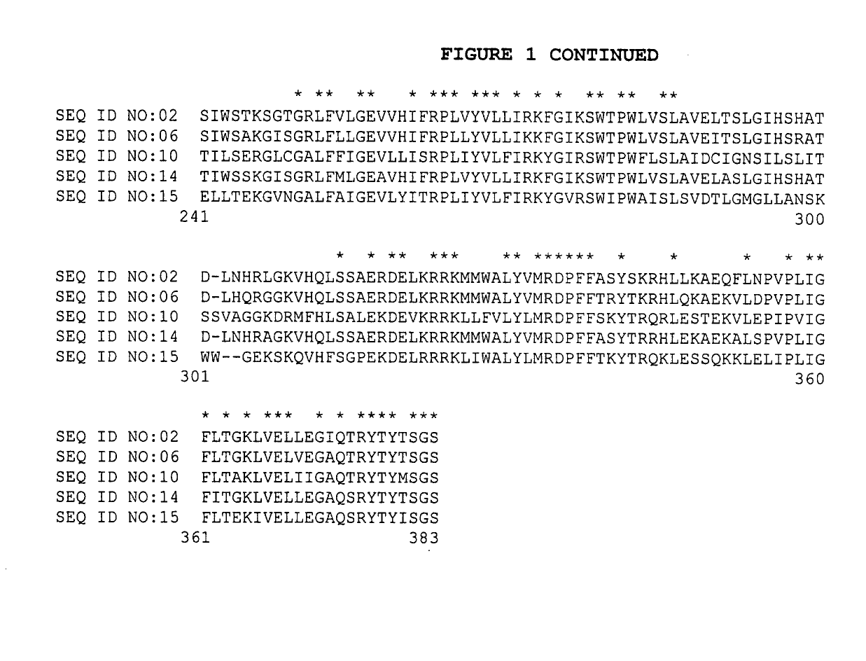

FIG. 1 depicts the amino acid sequence alignment between the SSE1 protein encoded by the nucleotide sequences derived from maize clone p0002.cgevh96r (SEQ ID NO: 2), rice clone r10n.pk0031.h7 (SEQ ID NO: 6), contig assembled from soybean clones sdp3c.pk008.c9 and pP54/pP55 (SEQ ID NO: 10), wheat clone wdk5c.pk0002.b10 (SEQ ID NO: 14), and the SSE1 gene from Arabidopsis thaliana (NCBI GenBank Identifier (GI) No. 4837733, SEQ ID NO: 15). The data in Table 4 represents a calculation of the percent identity of the amino acid sequences set forth in SEQ ID NOs: 2, 6, 10 and 14 and the Arabidopsis thaliana sequence (SEQ ID NO: 15).

TABLE 4
Percent Identity of Amino Acid Sequences
Deduced From the Nucleotide Sequences of cDNA
Clones Encoding Polypeptides Homologous to SSE1

| | Percent Identity to |
|---|---|
| SEQ ID NO. | 4837733 |
| 2 | 43.3 |
| 6 | 44.4 |
| 10 | 52.7 |
| 14 | 44.1 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of the coding region for SSE1 protein. These sequences represent the first soybean and monocot (maize, rice and wheat) sequences encoding SSE1 protein known to Applicant.

### Example 4

### Expression of Chimeric Genes in Monocot Cells

A chimeric gene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding the instant polypeptides, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35 S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains bialophos (5 mg per liter) and lacks casein or proline. The tissue continues to grow slowly on this medium. After an additional 2 weeks the tissue can be transferred to fresh N6 medium containing bialophos. After 6 weeks, areas of about 1 cm in diameter of actively growing callus can be identified on some of the plates containing the bialophos-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

Another method of transformation is by co-cultivation with Agrobacterium. Agrobacterium is streaked out from a −80° frozen aliquot onto a plate containing PHI-L medium and cultured at 28° C. in the dark for 3 days. PHI-L media comprises 25 ml/l Stock Solution A, 25 ml/l Stock Solution B, 450.9 ml/l Stock Solution C and spectinomycin (Sigma Chemicals) added to a concentration of 50 mg/l in sterile ddH₂O (stock solution A: K₂HPO₄ 60.0g/l, NaH₂PO₄ 20.0 g/l, adjust pH to 7.0 w/KOH and autoclave; stock solution B: NH₄Cl 20.0 g/l, MgSO₄.7H₂O 6.0 g/l, KCl 3.0 g/l, CaCl₂ 0.20 g/l, FeSO₄.7H₂O 50.0 mg/l, autoclave; stock solution C: glucose 5.56 g/l, agar 16.67 g/l (#A-7049, Sigma Chemicals, St. Louis, Mo.) and autoclave).

The plate can be stored at 4° C. and used usually for about 1 month. A single colony is picked from the master plate and streaked onto a plate containing PHI-M medium [yeast extract (Difco) 5.0 g/l; peptone (Difco) 10.0 g/l; NaCl 5.0 g/l; agar (Difco) 15.0 g/l; pH 6.8, containing 50 mg/L spectinomycin] and incubated at 28° C. in the dark for 2 days. Five ml of either PHI-A, [CHU(N6) basal salts (Sigma C-1416) 4.0 g/l, Eriksson's vitamin mix (1000X, Sigma-1511) 1.0 ml/l; thiamine.HCl 0.5 mg/l (Sigma); 2,4-dichlorophenoxyacetic acid (2,4-D, Sigma) 1.5 mg/l; L-proline (Sigma) 0.69 g/l; sucrose (Mallinckrodt) 68.5 g/l; glucose (Mallinckrodt) 36.0 g/l; pH 5.2] for the PHI basic medium system, or PHI-I [MS salts (GIBCO BRL) 4.3 g/l; nicotinic acid (Sigma) 0.5 mg/l; pyridoxine.HCl (Sigma) 0.5 mg/l; thiamine.HCl 1.0 mg/l; myo-inositol (Sigma) 0.10 g/l; vitamin assay casamino acids (Difco Lab) 1.0 g/l; 2, 4-D 1.5 mg/l; sucrose 68.50 g/l; glucose 36.0 g/l; adjust pH to 5.2 w/KOH and filter-sterilize] for the PHI combined medium system and 5 μl of 100 mM (3′-5′-dimethoxy-4′-hydroxyacetophenone, Aldrich chemicals) are added to a 14 ml Falcon tube in a hood. About 3 full loops (5 mm loop size) Agrobacterium is collected from the plate and suspended in the tube, then the tube is vortexed to make an even suspension. One ml of the suspension is transferred to a spectrophotometer tube and the OD of the suspension adjusted to 0.72 at 550 nm by adding either more Agrobacterium or more of the same suspension medium, for an Agrobacterium concentration of approximately 0.5×10⁹ cfu/ml to 1×10⁹ cfu/ml. The final Agrobacterium suspension is aliquoted into 2 ml microcentrifuge tubes, each containing 1 ml of the suspension. The suspensions are then used as soon as possible.

Embryo Isolation, Infection and Co-Cultivation

About 2 ml of the same medium (here PHI-A or PHI-I) used for the Agrobacterium suspension are added into a 2 ml microcentrifuge tube. Immature embryos are isolated from a sterilized ear with a sterile spatula (Baxter Scientific Products S1565) and dropped directly into the medium in the tube. A total of about 100 embryos are placed in the tube. The optimal size of the embryos is about 1.0-1.2 mm. The cap is then closed on the tube and the tube vortexed with a Vortex Mixer (Baxter Scientific Products S8223-1) for 5 sec. at maximum speed. The medium is removed and 2 ml of fresh medium are added and the vortexing repeated. All of the medium is drawn off and 1 ml of Agrobacterium suspension is added to the embryos and the tube vortexed for 30 sec. The tube is allowed to stand for 5 min. in the hood. The suspension of Agrobacterium and embryos was poured into a Petri plate containing either PHI-B medium [CHU(N6) basal salts (Sigma C-1416) 4.0 g/l; Eriksson's vitamin mix (1000X, Sigma-1511) 1.0 ml/l; thiamine.HCl 0.5 mg/l; 2.4-D 1.5 mg/l; L-proline 0.69 g/l; silver nitrate 0.85 mg/l; gelrite (Sigma) 3.0 g/l; sucrose 30.0 g/l; acetosyringone 100 μM; pH 5.8], for the PHI basic medium system, or PHI-J medium [MS Salts 4.3 g/l; nicotinic acid 0.50 mg/l; pyridoxine HCl 0.50 mg/l; thiamine.HCl 1.0 mg/l; myo-inositol 100.0 mg/l; 2, 4-D 1.5 mg/l; sucrose 20.0 g/l; glucose 10.0 g/l; L-proline 0.70 g/l; MES (Sigma) 0.50 g/l; 8.0 g/l agar (Sigma A-7049, purified) and 100 μM acetosyringone with a final pH of 5.8 for the PHI combined medium system. Any embryos left in the tube are transferred to the plate using a sterile spatula. The Agrobacterium suspension is drawn off and the embryos placed axis side down on the media. The plate is sealed with Parafilm tape or Pylon Vegetative Combine Tape (product named “E.G.CUT” and is available in 18 mm×50 m sections; Kyowa Ltd., Japan) and incubated in the dark at 23-25° C. for about 3 days of co-cultivation.

Resting, Selection and Regeneration Steps

For the resting step, all of the embryos are transferred to a new plate containing PHI-C medium [CHU(N6) basal salts (Sigma C-1416) 4.0 g/l; Eriksson's vitamin mix (1000X Sigma-1511) 1.0 ml/l; thiamine.HCl 0.5 mg/l; 2.4-D 1.5 mg/l; L-proline 0.69 g/l; sucrose 30.0 g/l; MES buffer (Sigma) 0.5 g/l; agar (Sigma A-7049, purified) 8.0 g/l; silver nitrate 0.85 mg/l; carbenicillin 100 mg/l; pH 5.8]. The plate is sealed with Parafilm or Pylon tape and incubated in the dark at 28° C. for 3-5 days.

Longer co-cultivation periods may compensate for the absence of a resting step since the resting step, like the co-cultivation step, provides a period of time for the embryo to be cultured in the absence of a selective agent. Those of ordinary skill in the art can readily test combinations of co-cultivation and resting times to optimize or improve the transformation frequency of other inbreds without undue experimentation.

For selection, all of the embryos are then transferred from the PHI-C medium to new plates containing PHI-D medium, as a selection medium, [CHU(N6) basal salts (SIGMA C-1416) 4.0 g/l; Eriksson's vitamin mix (1000X, Sigma-1511) 1.0 ml/l; thiamine.HCl 0.5 mg/l; 2.4-D 1.5 mg/l; L-proline 0.69 g/l; sucrose 30.0 g/l; MES buffer 0.5 g/l; agar (Sigma A-7049, purified) 8.0 g/l; silver nitrate 0.85 mg/l; carbenicillin (ICN, Costa Mesa, Calif.) 100 mg/l; bialaphos (Meiji Seika K.K., Tokyo, Japan) 1.5 mg/l for the first two weeks followed by 3 mg/l for the remainder of the time.; pH 5.8] putting about 20 embryos onto each plate. The plates are sealed as described above and incubated in the dark at 28° C. for the first two weeks of selection. The embryos are transferred to fresh selection medium at two-week intervals. The tissue is subcultured by transferring to fresh selection medium for a total of about 2 months. The herbicide-resistant calli are then “bulked up” by growing on the same medium for another two weeks until the diameter of the calli is about 1.5-2 cm.

For regeneration, the calli are then cultured on PHI-E medium [MS salts 4.3 g/l; myo-inositol 0.1 g/l; nicotinic acid 0.5 mg/l, thiamine.HCl 0.1 mg/l, Pyridoxine.HCl 0.5 mg/l, Glycine 2.0 mg/l, Zeatin 0.5 mg/l, sucrose 60.0 g/l, Agar (Sigma, A-7049) 8.0 g/l, Indoleacetic acid (IAA, Sigma) 1.0 mg/l, Abscisic acid (ABA, Sigma) 0.1 μM, Bialaphos 3 mg/l, carbenicillin 100 mg/l adjusted to pH 5.6] in the dark at 28° C. for 1-3 weeks to allow somatic embryos to mature. The calli are then cultured on PHI-F medium (MS salts 4.3 g/l; myo-inositol 0.1 g/l; Thiamine.HCl 0.1 mg/l, Pyridoxine.HCl 0.5 mg/l, Glycine 2.0 mg/l, nicotinic acid 0.5 mg/l; sucrose 40.0 g/l; gelrite 1.5 g/l; pH 5.6] at 25° C. under a daylight schedule of 16 hrs. light (270 uE m⁻²sec⁻¹) and 8 hrs. dark until shoots and roots develop. Each small plantlet is then transferred to a 25×150 mm tube containing PHI-F medium and grown under the same conditions for approximately another week. The plants are transplanted to pots with soil mixture in a greenhouse. GUS+ events are determined at the callus stage or regenerated plant stage.

For Hi-II a preferred optimized protocol was 0.5×109 cfu/ml Agrobacterium, a 3-5 day resting step, and no AgNO₃ in the infection medium (PHI-A medium). Hi-II is the F₁ of two purebred genetic lines, parent A and parent B, derived from A 188×B73. Both parents are selected for high competence of somatic embryogenesis. See Armstrong, et al., “Development and Availability of Germplasm with High Type II Culture Formation Response,” Maize Genetics Cooperation Newsletter, Vol. 65, pp. 92 (1991); incorporated herein in its entirety by reference. The examples provide a variety of experiments that similarly teach those of ordinary skill in the art to optimize transformation frequencies for other maize lines and other monocots.

### Example 5

### Expression of Chimeric Genes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC 18 vector carrying the seed expression cassette.

Soybean embroys may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70, U.S. Pat. No. 4,945,050). A DuPont Biolistic™ PDS 1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Expression of Chimeric Genes in Microbial Cells

The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% low melting agarose gel. Buffer and agarose contain 10 μg/ml ethidium bromide for visualization of the DNA fragment. The fragment can then be purified from the agarose gel by digestion with GELase™ (Epicentre Technologies, Madison, Wis.) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

What is claimed is:

1. An isolated polynucleotide comprising a nucleotide sequence encoding a polypeptide comprising at least 50 amino acids, wherein the amino acid sequence of the polypeptide and SEQ ID NO: 2, SEQ ID NO: 4, SEQ ID NO: 6, SEQ ID NO: 8, SEQ ID NO: 10, SEQ ID NO: 12, or SEQ ID NO: 14 have at least 80% identity based on the Clustal alignment method.

2. The isolated polynucleotide of claim 1, wherein the polypeptide comprises 100 amino acids.

3. The isolated polynucleotide of claim 1, wherein the polypeptide comprises SEQ ID NO: 2, SEQ ID NO: 4, SEQ ID NO: 6, SEQ ID NO: 8, SEQ ID NO: 10, SEQ ID NO: 12, or SEQ ID NO: 14.

4. The isolated polynucleotide of claim 1, wherein the nucleotide sequence comprises SEQ ID NO: 1, SEQ ID NO: 3, SEQ ID NO: 5, SEQ ID NO: 7, SEQ ID NO: 9, SEQ ID NO: 11, or SEQ ID NO: 13.

5. The isolated polynucleotide of claim 1, wherein the polypeptide is a SSE1 protein.

6. The complement of the polynucleotide of claim 1, wherein the complement and the polynucleotide consist of the same number of nucleotides and are 100% complementary.

7. An isolated polypeptide encoded by the nucleotide sequence comprised by the polynucleotide of claim 1.

8. A method for transforming a cell comprising introducing the polynucleotide of claim 1 into a cell.

9. The cell produced by the method of claim 8.

10. A method for transforming a cell comprising introducing the complement of claim 6 into a cell.

11. The cell produced by the method of claim 10.

12. A polynucleotide fragment comprising a nucleotide sequence comprised by the polynucleotide of claim 1, wherein the nucleotide sequence contains at least 30 nucleotides.

13. The polynucleotide fragment of claim 12, wherein the nucleotide sequence contains at least 40 nucleotides.

14. The polynucleotide fragment of claim 12, wherein the nucleotide sequence contains at least 60 nucleotides.

15. A polynucleotide fragment comprising a nucleotide sequence comprised by the complement of claim 6, wherein the nucleotide sequence contains at least 30 nucleotides.

16. The polynucleotide fragment of claim 15, wherein the nucleotide sequence contains at least 40 nucleotides.

17. The polynucleotide fragment of claim 15, wherein the nucleotide sequence contains at least 60 nucleotides.

18. A transgenic plant comprising in its genome a chimeric gene comprising the polynucleotide of claim 1.

19. The transgenic plant of claim 18, wherein the plant is maize, soybean, alfalfa, sunflower, canola, cotton, palm, flax, sorghum, wheat, barley, millet or rice.

20. A seed from the transgenic plant of claim 19.

21. The seed of claim 20, wherein the seed is from maize, soybean, alfalfa, sunflower, canola, cotton, palm, flax, sorghum, wheat, barley, millet or rice.

22. A method for modulating the level of SSE1 in a plant, comprising:
(a) stably transforming a plant cell with an SSE1 polynucleotide operably linked to a promoter, wherein the polynucleotide is in sense or antisense orientation;
(b) growing the plant cell under plant growing conditions to produce a regenerated plant capable of expressing the polynucleotide for a time sufficient to modulate the level of SSE1 in the plant.

23. The method of claim 22, wherein the polynucleotide is selected from those of claim 1.

24. The method of claim 22, wherein SSE1 level is reduced to result in an increase in starch deposition in the endosperm.

25. The method of claim 22, wherein SSE1 level is increased to result in an increase in oil deposition in the embryo.

26. The method of claim 22, wherein SSE1 level is increased to result in an increase in protein content in the seed.

27. The method of claim 22, wherein SSE1 level is increased to result in an increase in oil and protein content in the seed.

28. The method of claim 22, wherein the plant is maize, soybean, alfalfa, sunflower, canola, cotton, palm, flax, sorghum, wheat, barley, millet, or rice.

29. A method for modulating the relative amounts of oil, protein, and/or starch in the seed of a plant, comprising:
(a) stably transforming a plant cell with an SSE1 polynucleotide operably linked to a promoter, wherein the polynucleotide is in sense or antisense orientation;
(b) growing the plant cell under plant growing conditions to produce a regenerated plant capable of expressing the polynucleotide for a time sufficient to modulate the relative amounts of oil, protein, and/or starch in the seed.

30. The method of claim 29, wherein the polynucleotide is selected from those of claim 1.

31. The method of claim 29, wherein the plant is maize, soybean, alfalfa, sunflower, canola, cotton, palm, flax, sorghum, wheat, barley, millet, or rice.

32. A method for modulating storage organ formation in the seed of a plant, comprising:
(a) stably transforming a plant cell with an SSE1 polynucleotide operably linked to a promoter, wherein the polynucleotide is in sense or antisense orientation;
(b) growing the plant cell under plant growing conditions to produce a regenerated plant capable of expressing the polynucleotide for a time sufficient to modulate storage organ formation in the seed.

33. The method of claim 32, wherein the polynucleotide is selected from those of claim 1.

34. The method of claim 32, wherein the plant is maize, soybean, alfalfa, sunflower, canola, cotton, palm, flax, sorghum, wheat, barley, millet, or rice.

35. A method for improving the food, feed, and/or industrial processing value of grain, comprising:
(a) stably transforming a plant cell with an SSE1 polynucleotide operably linked to a promoter, wherein the polynucleotide is in sense or antisense orientation;
(b) growing the plant cell under plant growing conditions to produce a regenerated plant capable of expressing the polynucleotide for a time sufficient to improve the food, feed, and/or industrial processing value of the grain produced by the plant.

36. The method of claim 35, wherein the polynucleotide is selected from those of claim 1.

37. The method of claim 35, wherein the plant is maize, soybean, alfalfa, sunflower, canola, cotton, palm, flax, sorghum, wheat, barley, millet, or rice.

38. A method for providing plants capable of partitioning photosynthate to produce seed with improved functional properties for use in specific food and non-food industrial applications, comprising:
(a) stably transforming a plant cell with an SSE1 polynucleotide operably linked to a promoter, wherein the polynucleotide is in sense or antisense orientation;
(b) growing the plant cell under plant growing conditions to produce a regenerated plant capable of expressing the polynucleotide for a time sufficient to partition photosynthate to produce seed with improved functional properties.

39. The method of claim 38, wherein the polynucleotide is selected from those of claim 1.

40. The method of claim 38, wherein the plant is maize, soybean, alfalfa, sunflower, canola, cotton, palm, flax, sorghum, wheat, barley, millet, or rice.


SEQUENCE LISTING (18 sequences)

SEQ ID NO: 1 - DNA, 1707 bp, Zea mays
gaattcggcacgagcgccaatcctccccatcccgaccgccgcccccacctcctccgcggccgcctcgcggcgattccgtccgcatctgaccccgatcccaggggcatcgtcctcacctcctccgccagctgccgctcttccgtcctcttcccagcttctgcgtggggaagaggcggtggcggccgaaccggcgtagtcgtcgccgacgccgctgccagccgccatggaggcgtacaagctctgggtgcgcaggaacagggacctcgtccgctccctcgagtccctcgccaacgggctcacatggatactccccgagcgcttcgccaactccgagatcgcaccagaagcagtatatgcactactgggtattgtgagttctgtcaatcagcacataattgatgcgcccactgagaatcactcatttgcctccaaggaacaatctatcccatggggtcttgttgtctctgtactaaaggatgtggaggcggttgttgaggttgctgcccagcactttgttggcgatgatcgcaagtggagctttcttgctgttacagaagcagtgaaagcaggtgtcaggttagctgcttttcgggagagtggatacaagatgctcttacaaggaggggaggtggtaaatgaagaagaggtgaccgttcttgaaaataattatggagtaaatggtaatggagtaccagccatctatccgatggatggacatgcagaaaatggtcacaaaactatggccaagggtctggatggtaaaaatggatttgtatctaagagtcttgagaaaagagcagtagctgctttgaacaaatttggtgagaacgcaaagatgatgtctgatcctatgtggatgcggaggccccaacctactcctgagccaactgtgatggttgccgagaagccaacattgacaagtatttggtctactaaaagcggtactgggcgcttgtttgttttaggggaggttgttcacatattcaggccacttgtatatgtacttctgatcagaaagtttggaatcaaatcatggaccccgtggctagtgtcgctagctgtggaactcacaagtctaggcatccattcccatgcaaccgatctgaatcacagattagggaaagtgcatcagctcagttctgccgaaagggacgagttgaaaaggcgaaagatgatgtgggctctttatgtgatgagagatcctttctttgccagttacagcaagcgtcacctcctgaaggctgaacagtttctgaatccggtgccattgattggcttccttacagggaaacttgtagagctactggaggggattcagacgagatacacgtacacatcaggttcatagagatggccaatctgagcctgctgctcgcctctcgatttgccctggcggacatgtgctttgtgcgagttggttgatggttaatggttaatggctaggaactggtgcttaatcctgaaacccgtactgctgtttttcttgccaactgtggcatcgtcgtcttgtggctgcgaagctgcagccacctcgttcgtgtatggcggcagatgagacaattcataatctaagtatatagatataaatagtagtattaccggtttgtttgtatttacgatttatcgtgaactgatggaacataatgtgtatacagcgaaaatttatctgattccaaacatttgttgtttaaaaaaaaaaaaaaaaaa

SEQ ID NO: 2 - protein, 374 aa, Zea mays
MEAYKLWVRRNRDLVRSLESLANGLTWILPERFANSEIAPEAVYALLGIVSSVNQHIIDAPTENHSFASKEQSIPWGLVVSVLKDVEAVVEVAAQHFVGDDRKWSFLAVTEAVKAGVRLAAFRESGYKMLLQGGEVVNEEEVTVLENNYGVNGNGVPAIYPMDGHAENGHKTMAKGLDGKNGFVSKSLEKRAVAALNKFGENAKMMSDPMWMRRPQPTPEPTVMVAEKPTLTSIWSTKSGTGRLFVLGEVVHIFRPLVYVLLIRKFGIKSWTPWLVSLAVELTSLGIHSHATDLNHRLGKVHQLSSAERDELKRRKMMWALYVMRDPFFASYSKRHLLKAEQFLNPVPLIGFLTGKLVELLEGIQTRYTYTSGS

SEQ ID NO: 3 - DNA, 429 bp, Oryza sativa
  unsure (112)
  unsure (126)
  unsure (155)
  unsure (266)
  unsure (273)
  unsure (320)
  unsure (396)..(397)
  unsure (417)
cttacagatagccgaagccgaagccgaagccgccctgctctgacacctcgattcaccccgcctcgccgccggccaccgccgccgcagatcaggcggctccagcgggcggtanggctcctcctgtangaggagttggtgggtaccgtcgcgttctnctttcccctagctaggtctcgccagaaggaggaggaggcggtggctgcggcgcggtcgccatggaggcatacaagctctgggtgcgcaagaaccgggacctcgtccgctcnctcgagncgttggccaatgggctaacgtggatacttcctgagcgctttgccaantctgagatcgcaccagaagcagtatatgcatttctgggtatcgtgagttctgtcaatcagcacataattgaaacgnnactgattgtagacattgggnctcaaagaggca

SEQ ID NO: 4 - protein, 59 aa, Oryza sativa
  UNSURE (20)
  UNSURE (35)
MEAYKLWVRKNRDLVRSLEXLANGLTWILPERFAXSEIAPEAVYAFLGIVSSVNQHIIE

SEQ ID NO: 5 - DNA, 2320 bp, Oryza sativa
gcacgagcttacagatagccgaagccgaagccgaagccgccctgctctgacacctcgattcaccccgcctcgccgccggccaccgccgccgcagatcaggcggctccagcgggcggtagggctcctcctgtaggaggagttggtgggtaccgtcgcgttcttctttcccctagctaggtctcgccagaaggaggaggaggcggtggctgcggcgcggtcgccatggaggcatacaagctctgggtgcgcaagaaccgggacctcgtccgctccctcgagtcgttggccaatgggctaacgtggatacttcctgagcgctttgccaactctgagatcgcaccagaagcagtatatgcatttctgggtatcgtgagttctgtcaatcagcacataattgaaacgccaactgatggtcagacattggcctccaaagagcaatctatcccatggtcccttgttgtctcagtacttaaggatattgaggcagttgttgaggtggctgcccagcactttgttggagatgatcgcaaatggagctttcttgctgttacagaagctgtgaaagcaggtgtcaggttagctgctttcggggagagtggctacaagatgctcttacaaggaggagaggtggcaaatgaagaggagattaatattcttgatgaaaattttggagccaaaagtaatggagtaccagtcatttatccgatgaatggccatttccaaaatggtcatggggttgcatctaatggtcttgatggaaaggctggatttgtatcaaagagtctggagggaagagctgtagctgctcttaacaagtttggccagaatgcaaagatgacgtcagatcccatgtggatgaagaaggctctgcctcctcctgatcctcctgcgatggtggttgagaagccaactttggcaagtatttggtctgctaaaggaatttcagggcggttatttttgttaggagaagttgtccacatattcagaccactgctatacgtacttttgatcaaaaaatttggaatcaaatcatggaccccatggttagtgtcattagctgtggagatcacaagtcttggcatccattcacgtgcaactgatcttcatcaaagagggggaaaagttcatcagctctcatctgctgagagggacgagttgaaaaggcgaaagatgatgtgggccctttatgtcatgagagatccattctttaccagatacaccaagcgccatctccagaaggctgagaaagtgttggatccagtgcctcttattggtttccttacaggcaaactcgtagagctagtggagggggctcagacacgatatacatacacatcgggctcataaggataatggacaagcaggcagatgtcatgtctgagagtttccttaacgatttgccatgattaaccttttgtggttcatgtgatctggttgatgggttttgttttgagcttgatcctaatcctatctgtacatgccatttccttagcaagatttggcattcctacctgatgtcttggggctgcaaagctgttgcggccactgtaaactctcctctctcctctggtggcagctcagctgagaatctaactatactatagtatatcggtgtaatattaagaataggcacatcatccctgcaacgataccgtgtatttatttaccaattaccatgcaggacatactggaacccaaaaaaaaaaaaaaaaactcgagaccgagcagcagcagcagagcttagcagcattccatggcgatctcctccgtcttcctgcgtccatcgctgttctcttccccgccggcggcggcggcggcttcctctccccggcgacatgcagcagtgctacgcgtcacctcgagcaagaggagaccactcttctcaagggcggcgacgtcgctgacggtgagatgcgagcagacggcgaagccaggcggcggcaccggcgccggcgccgccgacgtgtggctgagccgcctcgccatggtcagcttctccaccgccgtcgtcgtcgaggtctccaccggcgaaggcctcgtcgcgaacttgggcgtggcgacgccggcgccgacgctggcgctggtggtgacgtcactcgccgccggcctcgccgtctacttcatcttccaggccggctcccgcaactgaagaaacaaaccgaactgaatcgctgaaacatccaagaacttgacatctcaacatgttcttctcaactgatgatgagaattaagattattatctctgggatcggactagttcttgcaaatatacaagcatatatagaaatgatgattgatgacaaaaaaaaaaaaaaaaaaa

SEQ ID NO: 6 - protein, 374 aa, Oryza sativa
MEAYKLWVRKNRDLVRSLESLANGLTWILPERFANSEIAPEAVYAFLGIVSSVNQHIIETPTDGQTLASKEQSIPWSLVVSVLKDIEAVVEVAAQHFVGDDRKWSFLAVTEAVKAGVRLAAFGESGYKMLLQGGEVANEEEINILDENFGAKSNGVPVIYPMNGHFQNGHGVASNGLDGKAGFVSKSLEGRAVAALNKFGQNAKMTSDPMWMKKALPPPDPPAMVVEKPTLASIWSAKGISGRLFLLGEVVHIFRPLLYVLLIKKFGIKSWTPWLVSLAVEITSLGIHSRATDLHQRGGKVHQLSSAERDELKRRKMMWALYVMRDPFFTRYTKRHLQKAEKVLDPVPLIGFLTGKLVELVEGAQTRYTYTSGS

SEQ ID NO: 7 - DNA, 1505 bp, Glycine max
  unsure (59)
  unsure (60)
  unsure (90)
  unsure (93)
  unsure (107)
  unsure (109)
  unsure (207)
  unsure (251)
  unsure (266)
  unsure (342)
  unsure (353)
  unsure (419)
  unsure (459)
  unsure (542)
  unsure (558)
  unsure (575)
  unsure (578)
  unsure (590)
  unsure (650)
  unsure (661)
  unsure (675)
  unsure (684)
  unsure (686)
  unsure (706)
  unsure (707)
  unsure (713)
  unsure (720)
  unsure (742)
gccgcgggaattcgattctctagaactagtggatcccccgggctgcaggaattcggackrggtcgcttccaataccttcagattttggtwggrtttcgtgcttttgsawaattcgttgagtttctgaagctatggaggcttataagagatgggtgaggcagaacaaagagtttgtgcactccatggagtctttggccaatggattgrcatggcttcttcctgaacggttttctgaatcagagattggaccwgaagcagtaacaacyattctgggaatcatcacagctctcaatgaacatataattgatacagctcctaagcaaaatattacaggctctgtcragccttattcrtttccttatccattatgcttatctgcattaaaggatttggaaacattagttgaagttgtggcacarcaatactatggtgatgataagaaatggaatttccttgctrttactgaagcaaccaaggtactggttcggttatctttgtttcggaagagtggatataagatgctgctacaaggaggggaaacwcctaatgatgaggagyattcagatagttttacytcrcaacatcatatrggcttaaagcccgatgtgcatcataggcctggttatatgaaaaacaatcttggtgcaaamccaatgaatcwggaaggaagagcaytatctgctytrgttagatttggagaaaaagyraagggrtcagatycagtgtggttacgcagggttgracaccaacaagcaactatggagcctacaacttcaagggtagatagaccaacacttctcaccatattgtctgaaaggggtctttgtggggctctgttttttattggagaagttctacttattagtagaccacttatttatgttttatttattcgaaaatatggtattcggtcatggaccccttggttcctttcgctggctattgattgcataggaaacagtattctttcactcattacatcgtcagtggctggtgggaaggaccgaatgtttcatctgtctgccctagaaaaggatgaggttaaacggcgaaagctgctatttgttctttacctaatgagagatccatttttcagcaagtatactaggcaaagacttgaaagcacggagaaagttttggagcctattcctgtcataggatttctcacagcaaaacttgttgaacttataattggagctcaaacacgatacacttacatgtcaggatcgtgaataaaatccagaacaaatgcctaattgccctccaagattttggaaagatagatattcttactcttcttccacactacctgctgttccaaacttttcaaatgatgaagaggtatcaaacctgctactattatgatttaaaaataactaaccattgcaagcttgaacttttcttttgcttgacaattccaaacatagaagatgttaagctgccacccatgtgtgaggcaaattgtttgcaaggatagctacactatcaacaactcagtatgaatt

SEQ ID NO: 8 - protein, 366 aa, Glycine max
  UNSURE (26)
  UNSURE (71)
  UNSURE (110)
  UNSURE (143)
  UNSURE (153)
  UNSURE (173)
  UNSURE (177)
  UNSURE (192)
  UNSURE (197)
  UNSURE (204)
MEAYKRWVRQNKEFVHSMESLANGLXWLLPERFSESEIGPEAVTTILGIITALNEHIIDTAPKQNITGSVXPYSFPYPLCLSALKDLETLVEVVAQQYYGDDKKWNFLAXTEATKVLVRLSLFRKSGYKMLLQGGETPNDEEXSDSFTSQHHXGLKPDVHHRPGYMKNNLGAXPMNXEGRALSALVRFGEKXKGSDXVWLRRVXHQQATMEPTTSRVDRPTLLTILSERGLCGALFFIGEVLLISRPLIYVLFIRKYGIRSWTPWFLSLAIDCIGNSILSLITSSVAGGKDRMFHLSALEKDEVKRRKLLFVLYLMRDPFFSKYTRQRLESTEKVLEPIPVIGFLTAKLVELIIGAQTRYTYMSGS

SEQ ID NO: 9 - DNA, 1505 bp, Glycine max
  unsure (59)
  unsure (60)
  unsure (90)
  unsure (93)
  unsure (107)
  unsure (109)
  unsure (207)
  unsure (251)
  unsure (266)
  unsure (342)
  unsure (353)
  unsure (419)
  unsure (459)
  unsure (542)
  unsure (558)
  unsure (575)
  unsure (578)
  unsure (590)
  unsure (650)
  unsure (661)
  unsure (675)
  unsure (684)
  unsure (686)
  unsure (706)
  unsure (707)
gccgcgggaattcgattctctagaactagtggatcccccgggctgcaggaattcggackrggtcgcttccaataccttcagattttggtwggrtttcgtgcttttgsawaattcgttgagtttctgaagctatggaggcttataagagatgggtgaggcagaacaaagagtttgtgcactccatggagtctttggccaatggattgrcatggcttcttcctgaacggttttctgaatcagagattggaccwgaagcagtaacaacyattctgggaatcatcacagctctcaatgaacatataattgatacagctcctaagcaaaatattacaggctctgtcragccttattcrtttccttatccattatgcttatctgcattaaaggatttggaaacattagttgaagttgtggcacarcaatactatggtgatgataagaaatggaatttccttgctrttactgaagcaaccaaggtactggttcggttatctttgtttcggaagagtggatataagatgctgctacaaggaggggaaacwcctaatgatgaggagyattcagatagttttacytcrcaacatcatatrggcttaaagcccgatgtgcatcataggcctggttatatgaaaaacaatcttggtgcaaamccaatgaatcwggaaggaagagcaytatctgctytrgttagatttggagaaaaagyraaggggtcagatccagtgtggttacgcagggttgaacaccaacaagcaactatggagcctacaacttcaagggtagatagaccaacacttctcaccatattgtctgaaaggggtctttgtggggctctgttttttattggagaagttctacttattagtagaccacttatttatgttttatttattcgaaaatatggtattcggtcatggaccccttggttcctttcgctggctattgattgcataggaaacagtattctttcactcattacatcgtcagtggctggtgggaaggaccgaatgtttcatctgtctgccctagaaaaggatgaggttaaacggcgaaagctgctatttgttctttacctaatgagagatccatttttcagcaagtatactaggcaaagacttgaaagcacggagaaagttttggagcctattcctgtcataggatttctcacagcaaaacttgttgaacttataattggagctcaaacacgatacacttacatgtcaggatcgtgaataaaatccagaacaaatgcctaattgccctccaagattttggaaagatagatattcttactcttcttccacactacctgctgttccaaacttttcaaatgatgaagaggtatcaaacctgctactattatgatttaaaaataactaaccattgcaagcttgaacttttcttttgcttgacaattccaaacatagaagatgttaagctgccacccatgtgtgaggcaaattgtttgcaaggatagctacactatcaacaactcagtatgaatt

SEQ ID NO: 10 - protein, 366 aa, Glycine max
  UNSURE (26)
  UNSURE (71)
  UNSURE (110)
  UNSURE (143)
  UNSURE (153)
  UNSURE (173)
  UNSURE (177)
  UNSURE (192)
MEAYKRWVRQNKEFVHSMESLANGLXWLLPERFSESEIGPEAVTTILGIITALNEHIIDTAPKQNITGSVXPYSFPYPLCLSALKDLETLVEVVAQQYYGDDKKWNFLAXTEATKVLVRLSLFRKSGYKMLLQGGETPNDEEXSDSFTSQHHXGLKPDVHHRPGYMKNNLGAXPMNXEGRALSALVRFGEKXKGSDPVWLRRVEHQQATMEPTTSRVDRPTLLTILSERGLCGALFFIGEVLLISRPLIYVLFIRKYGIRSWTPWFLSLAIDCIGNSILSLITSSVAGGKDRMFHLSALEKDEVKRRKLLFVLYLMRDPFFSKYTRQRLESTEKVLEPIPVIGFLTAKLVELIIGAQTRYTYMSGS

SEQ ID NO: 11 - DNA, 461 bp, Triticum aestivum
  unsure (328)
  unsure (350)
  unsure (387)
  unsure (435)
  unsure (442)
  unsure (450)
gtcgccgcggccggccaccgcgattcaccgccgccgtccgccggagatcggacggctgtccctgccccgacgccgtccctcgcgagccagtgccgccgcctcgtcctcatcgccgtcgccgccttcctcttcttccacccctagcttctccagaggcggccgagtcgccgagtcgccatggaggcctacaaggtctgggtgcggaagaaccgggacctcgtccgctccctcgagtccctcgccaacggggtgacatggatacttcctgagcgcttcgctaactccgagattgccccggaagcagtatatgcattctggggattgtaantctgtcaaccaacatataattngagacaccaaactgatgggcatcaatgggctccaaanggacaatctatccaatgggtcttgttgtatctatatccaaggattccnaacattnttgaattnccgccaacact

SEQ ID NO: 12 - protein, 49 aa, Triticum aestivum
MEAYKVWVRKNRDLVRSLESLANGVTWILPERFANSEIAPEAVYAFWGL

SEQ ID NO: 13 - DNA, 1468 bp, Triticum aestivum
gcaccaggtcgccgcggccggccaccgcgattcaccgccgccgtccgccggagatcggacggctgtccctgccccgacgccgtccctcgcgagccagtgccgccgcctcgtcctcatcgccgtcgccgccttcctcttcttccacccctagcttctccagaggcggccgagtcgccgagtcgccatggaggcctacaaggtctgggtgcggaagaaccgggacctcgtccgctccctcgagtccctcgccaacggggtgacatggatacttcctgagcgcttcgctaactccgagattgccccggaagcagtatatgcacttctgggcattgtaagttctgtcaaccagcatataattgagacaccaactgatggtcactcactggcctccaaggaacaatctatcccatgggctcttgttgtatctatactcaaggatgtcgaagcagttgttgaagttgccgcccagcactttgttggagatgatcgcaaatggggcttccttgctgttacagaagcagtgaaagcatgtgtcaggttagccgctttcagggagaatggctacaggatgctcctacaaggaggggaggtggaaaacgaagaggaggatgttcttgaagacaatcagggagtcaagactaatggagtgccagtaatctatccggtcaatggacattcccaaaatggccattggatcatgtctgatggtccggatggaaaacctggaattatatctaagactctggagggaagagcagtagctgctttaaacaggtttggtcagaatgcaaagatgttgtcagatcccacgtggatgagcaggctccaaccttctcctgttcctcctgtgatggagattgagaagccaactctcgcaaccatttggtcttctaaagggatttctgggcgcttattcatgttaggggaggccgtccacatattcagaccacttgtatacgtactcttgattagaaagtttggcatcaaatcttggaccccgtggttggtctcactagctgtggagctcgcaagccttggcattcattcgcatgcaacagatctgaatcatagagctgggaaagttcatcagctctcgtctgctgagagggatgagttgaaaaggcgaaaaatgatgtgggcactttatgtcatgagagatccattctttgccagctacaccaggcgtcatcttgagaaggctgagaaagcacttagtccggtgccgcttatcggtttcatcacaggtaaactcgtggaactattggagggggctcagtcgcggtatacatatacatcagggtcgtagaggaggattgggatagatttacctgcttctgctggagagcttccttgctgatctgccatactggacttttgctggttcctggattttgctttcagtagatgaggatttgagcgaaaccctgtctttgctttgccatttcgtagccagatctggcatcgctgt

SEQ ID NO: 14 - protein, 373 aa, Triticum aestivum
MEAYKVWVRKNRDLVRSLESLANGVTWILPERFANSEIAPEAVYALLGIVSSVNQHIIETPTDGHSLASKEQSIPWALVVSILKDVEAVVEVAAQHFVGDDRKWGFLAVTEAVKACVRLAAFRENGYRMLLQGGEVENEEEDVLEDNQGVKTNGVPVIYPVNGHSQNGHWIMSDGPDGKPGIISKTLEGRAVAALNRFGQNAKMLSDPTWMSRLQPSPVPPVMEIEKPTLATIWSSKGISGRLFMLGEAVHIFRPLVYVLLIRKFGIKSWTPWLVSLAVELASLGIHSHATDLNHRAGKVHQLSSAERDELKRRKMMWALYVMRDPFFASYTRRHLEKAEKALSPVPLIGFITGKLVELLEGAQSRYTYTSGS

SEQ ID NO: 15 - protein, 367 aa, Arabidopsis thaliana
MEAYKQWVWRNREYVQSFGSFANGLTWLLPEKFSASEIGPEAVTAFLGIFTTINEHIIENAPTPRGHVGSSGNDPSLSYPLLIAILKDLETVVEVAAEHFYGDKKWNYIILTEAMKAVIRLALFRNSGYKMLLQGGETPNEEKDSNQSESQNRAGNSGRNLGPHGLGNQNHHNPWNLEGRAMSALSSFGQNARTTTSSTPGWSRRIQHQQAVIEPPMIKERRRTMSELLTEKGVNGALFAIGEVLYITRPLIYVLFIRKYGVRSWIPWAISLSVDTLGMGLLANSKWWGEKSKQVHFSGPEKDELRRRKLIWALYLMRDPFFTKYTRQKLESSQKKLELIPLIGFLTEKIVELLEGAQSRYTYISGS

SEQ ID NO: 16 - DNA, 36 bp, Artificial Sequence
  Description of Artificial Sequence Synthetic oligonucleotide
tcgacccacgcgtccgaaaaaaaaaaaaaaaaaaaa

SEQ ID NO: 17 - DNA, 20 bp, Artificial Sequence
  Description of Artificial Sequence Synthetic oligonucleotide
gcagacagatgaaacattcg

SEQ ID NO: 18 - DNA, 20 bp, Artificial Sequence
  Description of Artificial Sequence Synthetic oligonucleotide
ctctagaactagtggatccc